(19) United States
(12) Patent Application Publication
(10) Pub. No.: US 2021/0008230 A1
(43) Pub. Date: Jan. 14, 2021

(54) TARGETED MICROBUBBLE, PREPARATION METHOD THEREOF, AND USE THEREOF

(71) Applicant: THE THIRD AFFILIATED HOSPITAL OF SUN YAT-SEN UNIVERSITY, Guangzhou, Guangdong (CN)

(72) Inventor: Qiquan Sun, Guangzhou, Guangdong (CN)

(21) Appl. No.: 16/968,766

(22) PCT Filed: Apr. 26, 2018
(86) PCT No.: PCT/CN2018/084611

§ 371 Date: Aug. 10, 2020

(30) Foreign Application Priority Data
Feb. 8, 2018 (CN) 201810131940.2

(51) Int. Cl. A61K 49/22; A61K 47/42; A61K 9/107; G16C 20/50; G16H 50/20; G16H 50/50

(52) U.S. Cl. CPC A61K 49/223; A61K 47/42; A61K 49/221; G16H 50/50; G16C 20/50; G16H 50/20; A61K 9/1075

(57) ABSTRACT

The invention provides a targeted microbubble comprising a microbubble composed of a shell and a gas encapsulated in the shell, the shell is conjugated with a C4d antibody or a C3d antibody. The targeted microbubble of the present invention is employed as contrast agent for the ultrasonic imaging of C4d or C3d deposited in renal and cardiac allografts. The occurrence of antibody-mediated rejection (AMR) can be accurately diagnosed via qualitative and quantitative analysis.

### FIELD OF THE INVENTION

The present invention relates to a targeted microbubble, preparation method thereof, and use thereof.

### BACKGROUND OF THE INVENTION

Renal transplantation and cardiac transplantation are the optimal therapies for kidney failure and heart failure. In recent years, due to the emergence of new immunosuppressive agents, the incidence of T cell-mediated rejection has significantly reduced, the short-term survival rate of the recipients has significantly increased. However, antibody-mediated rejection (AMR) has become a major factor affecting allograft survival. According to reports, the current 10-year survival rate of renal allografts is less than 50%, and at least 60% of renal allograft dysfunction is due to AMR. Similarly, in cardiac transplantation, the incidence of AMR is as high as 10% -20%. The incidence of AMR greatly increases the probability of allograft dysfunction.

C4d is the cleavage product of C4b in the classical complement pathway. It covalently binds to the surface of endothelial cells in blood vessels. When AMR occurs, an antigen-antibody complex can activate the complement system and produce a large amount of C4d. C4d is highly specific for AMR; although there are some reports and studies on C4d-negative AMR, C4d is still the best single marker for the diagnosis of AMR. At present, a common C4d detection method is to perform immunohistochemical and immunofluorescence staining on tissue biopsy specimens, followed by semi-quantitative analysis of the staining results. However, the acquisition of tissue specimens is an invasive procedure: for example, the regularly performed fine-needle aspiration may lead to serious complications. Furthermore, due to the limitation of the biopsy site, tissue changes in the whole organ are sometimes not fully reflected; this sampling error inevitably affects diagnosis. If there exists a non-invasive method for the quantitative detection of C4d, we would be able to acquire more comprehensive and accurate information when AMR occurs.

Compared with regular ultrasound (US), contrast-enhanced ultrasound has good sensitivity and specificity because contrast agents are employed to increase the contrast to the surrounding tissues. The recently developed targeted ultrasound imaging not only possesses the advantages of contrast-enhanced ultrasound, but could also detect biological activities at the cellular and molecular level. The value and safety of contrast-enhanced ultrasound in disease diagnosis have long been proven in many years of clinical practice. Targeted microbubbles designed for vascular endothelial growth factor receptor 2 (VEGFR2) have been used to observe the formation of new blood vessels in various tumor tissues, and have entered clinical trial. The mechanism of targeted ultrasound is first based on the basic structure of microbubbles (MBs): gas is encapsulated by surface phospholipids, allowing enhanced contrast in vivo. Then, after the microbubbles reach the tissue to be observed, ligands that are fixed on the microbubble surface specifically bind to the markers in the tissue. In this way, the microbubbles act in a targeted manner. During ultrasound imaging, the quantitative detection of the targeted microbubbles can be achieved by a destruction-replenishment mode (schematic diagram). At present, in researches of targeted ultrasound relating to allograft rejection, there are reports of using targeted microbubbles targeting intracellular adhesion molecule-1 (ICAM-1) and T-lymphocytes (CD3, CD4, and CD8) to diagnose acute cell-mediated rejection; however, the aforementioned studies do not solve the problems of AMR diagnosis. Considering the wide expression of C4d on the surface of capillary endothelial cells in the kidney and the heart during the occurrence of AMR, C4d could be a potential target for targeted ultrasound in the non-invasive detection of AMR.

### SUMMARY OF THE INVENTION

The present invention studies using C4d-targeted microbubbles as a contrast agent in the ultrasonic imaging of C4d deposition in renal and cardiac allografts in a rat model. The occurrence of acute AMR can be accurately diagnosed via qualitative and quantitative analysis. On this basis, the present invention provides a targeted microbubble, preparation method thereof, and use thereof.

To achieve the objective of the invention, the technical solution of the present invention is as follows: a targeted microbubble, the targeted microbubble comprises a microbubble composed of a shell and a gas encapsulated in the shell, the shell is conjugated with a C4d antibody or a C3d antibody.

Preferably, the shell is coated with streptavidin, the C4d antibody is a biotin-labeled antibody.

Preferably, a surface of the shell is coated with streptavidin, the C3d antibody is a biotin-labeled antibody.

Preferably, the C4d antibody or the C3d antibody is a fluorescence-labeled antibody.

Preferably, a diameter of the targeted microbubble is 1 μm to 10 μm.

Preferably, the diameter of the targeted microbubble is 1 μm to 4 μm. More preferably, the diameter of the targeted microbubble is 1.3 μm.

Preferably, the shell comprises at least one selected from the group consisting of a phospholipid, a protein, a lipid and a polymers. Preferably, the gas comprises at least one selected from the group consisting of perfluorocarbon, nitrogen, octafluoropropane and sulfur hexafluoride.

The invention provides a method for preparing the aforementioned targeted microbubble, the method comprises mixing and incubating a microbubble with a biotin-labeled C4d antibody or a biotin-labeled C3d antibody to obtain the targeted microbubble; the microbubble comprises a shell coated with streptavidin.

The invention provides use of the aforementioned targeted microbubble as a contrast agent in preparing diagnostic reagent or diagnostic reagent kit for AMR in a renal allograft, AMR in a cardiac allograft, AMR in a hepatic allograft, an autoimmune disease, cancer, or a kidney disease.

The invention provides a system for diagnosing AMR in a renal allograft, the system comprises:

a data-inputting module for inputting first ultrasound intensity data and second ultrasound intensity data into a model calculation module; wherein an ultrasonic signal generated by a microbubble attached to a vascular lumen through C4d or C3d binding and a free circulating microbubble is recorded as the first ultrasonic intensity data; within a beam height of an ultrasonic transducer, power of an ultrasonic pulse is increased to uniformly destruct a microbubble attached to a tissue and the free circulating microbubble, the free circulating microbubble is replenished until reaching an image saturation level; at 10 seconds after destruction, a second ultrasound imaging is performed, an ultrasonic signal at this point is recorded as the second ultrasound intensity data;

the model calculation module comprises a normalized intensity difference model for calculating normalized intensity difference according to the first ultrasonic intensity data, the second ultrasonic intensity data , the normalized intensity difference NID=(the first ultrasonic intensity data−the second ultrasound intensity data)/the first ultrasound intensity data);

a result-outputting module, in which a NID value of a normal kidney is set as a control, the AMR is diagnosed when a NID value of a patient's renal allograft is significantly greater than the NID value of the normal kidney.

The invention provides a system for diagnosing AMR in a cardiac allograft, the system comprises:

a data-inputting module for inputting first ultrasound intensity data and second ultrasound intensity data into a model calculation module; wherein an ultrasonic signal generated by a microbubble attached to a vascular cavity through C4d or C3d binding and a free circulating microbubble is recorded as the first ultrasonic intensity data; within a beam height of an ultrasonic transducer, power of an ultrasonic pulse is increased to uniformly destruct a microbubble attached to a tissue and the free circulating microbubble, the free circulating microbubble is replenished until reaching an imaging saturation level, at 10 seconds after destruction, a second ultrasound imaging is performed, and an ultrasonic signal at this point is recorded as the second ultrasound intensity data;

the model calculation module comprises a normalized intensity difference model for calculating normalized intensity difference result according to the first ultrasonic intensity data and the second ultrasonic intensity data, the normalized intensity difference NID=((the first ultrasonic intensity data−the second ultrasound intensity data)/the first ultrasound intensity data);

a result-outputting module, in which a NID value of a normal heart is set as a control, the AMR is diagnosed when a NID value of a patient's cardiac allograft is significantly greater than the NID value of the normal heart.

The invention provides a method for diagnosing AMR in a renal allograft, the method comprises using the aforementioned targeted microbubble as a contrast agent for diagnosis.

Preferably, the method comprises steps of:

(1) recording an ultrasonic signal generated by a microbubble attached to a vascular lumen through C4d or C3d binding and a free circulating microbubble as a first ultrasonic intensity data; increasing power of an ultrasonic pulse within a beam height of an ultrasonic transducer to uniformly destruct a microbubble attached to a tissue and the free circulating microbubble; then, replenishing the free circulating microbubble until reaching an image saturation level; performing a second ultrasound imaging at 10 seconds after destruction, recording an ultrasonic signal at this point as a second ultrasound intensity data;

(2) using a NID value of a normal kidney as a control, the AMR is diagnosed when a NID value of a patient's renal allograft is significantly greater than the NID value of the normal kidney; wherein normalized intensity difference NID=((the first ultrasound intensity data−the second ultrasound

Intensity data)/the first ultrasound intensity data).

The invention provides A method for diagnosing AMR in a cardiac allograft, the method comprises using the aforementioned targeted microbubble as a contrast agent for diagnosis.

Preferably, the method comprises steps of:

(1) recording an ultrasonic signal that is generated by a microbubble attached to a vascular lumen through C4d or C3d binding and a free circulating microbubble as a first ultrasonic intensity data; increasing power of an ultrasonic pulse within a beam height of an ultrasonic transducer to uniformly destruct a microbubble attached to a tissue and the free circulating microbubble; then, replenishing the free circulating microbubble until reaching an image saturation level, performing a second ultrasound imaging at 10 seconds after destruction recording an ultrasonic signal at this point as a second ultrasound intensity data;

(2) using a NID value of a normal heart as a control, the AMR is diagnosed when a NID value of the patient's cardiac allograft is significantly greater than the NID value of the normal heart; wherein normalized intensity difference NID=((the first ultrasound intensity data−the second ultrasound Intensity data)/the first ultrasound intensity data).

Antibody-mediated rejection (AMR) is the primary cause of renal allograft dysfunction. It also significantly increases the risk of allograft rejection and dysfunction in heart transplant recipients. At present, the diagnosis of AMR relies on allograft biopsy, which is an invasive procedure that may cause serious complications. C4d is specifically expressed in the interstitial vascular endothelial cells of the allografts with AMR, and is currently considered as the best single marker for the diagnosis of AMR. In the present invention, in the rat models of antibody-mediated renal and cardiac allograft rejection established, the diffuse expression of C4d in the interstitial blood vessels of the allografts can be detected on the 3rd day after the operation. In the present invention, C4d-targeted microbubbles were used as an ultrasound contrast agent, and their applicability and feasibility in the non-invasive diagnosis of AMR were explored. C4d-targeted microbubbles and control microbubbles were used in AMR rat models, and a destruction-replenishment approach was used to obtain imaging signals of the targeted area. Qualitative image analysis of C4d showed that compared with the control group, the imaging signal intensities of the targeted microbubbles group in renal and cardiac allografts were significantly enhanced. Additionally, quantitative analysis of C4d was performed. which showed that the normalized intensity difference (NID) of the C4d targeted microbubbles group was significantly higher than those of the control microbubbles group and the allograft control group: (28.0±3.8% vs 6.7±2.2% and 5.4±2.2%) and (26.7±3.0% vs 8.40±1.2% and 7.1±2.0%). These qualitative and quantitative evidences directly confirmed the feasibility of using C4d targeted ultrasound to diagnose renal and cardiac allografts with AMR. The research results of the present invention suggest that the C4d-targeted ultrasound imaging detection method is expected to be applied to clinical practice for novel, non-invasive diagnosis of AMR. C3d and C4d have the same significance in renal and cardiac allograft AMR and can both be used in the design of targeted microbubbles. Additionally, apart from renal and cardiac allografts with AMR, C3d and C4d are also expressed in hepatic allografts with AMR, certain autoimmune diseases, tumors, and kidney diseases. C4d-targeted microbubbles can also be used in novel, non-invasive diagnosis of these diseases.

### BRIEF DESCRIPTION OF DRAWINGS

FIG. 1 is a schematic diagram of a MicroMarker™ Target Ready microbubble.

FIG. 2 illustrates an experimental procedure for performing C4d-targeted ultrasound imaging of renal and cardiac allografts.

FIG. 3A and FIG. 3B illustrate C4d antibodies and control antibodies binding to the surface of microbubbles (MBs); biotinylated C4d was labeled with FITC and subsequently conjugated with MBs. FIG. 3A: schematic diagrams showing the binding rates of control antibodies and FITC-labeled C4d antibodies to microbubbles analyzed by flow cytometry. FIG. 3B: an intense fluorescence signal was observed in the MBs in fluorescence microscopy, indicating the binding of C4d antibodies to MBs.

FIG. 4A and FIG. 4B are an antibody-mediated renal allograft rejection model established by performing kidney transplantation 2 weeks after skin transplantation. FIG. 4A: changes in the levels of donor-specific antibodies (IgG and IgM) after skin transplantation. FIG. 4B: histological evaluation of the renal allograft 3 days after transplantation. Hematoxylin and eosin staining revealed peritubular capillary vasculitis, tubular damage, and hemorrhage. C4d staining revealed extensive C4d deposition. The syngeneic renal graft served as a control. (* P<0.05, ** P<0.01, *** P<0.001. ST: skin transplantation; KT: kidney transplantation).

FIG. 5A and FIG. 5B include C4d-targeted ultrasound (US) images and normalized intensity differences (NIDs) of different groups. FIG. 5A: representative ultrasound (US) images generated using C4d-targeted microbubbles (MB_{C4d}) and control microbubbles (MB_{Con}) in a syngeneic renal graft and a renal allograft with AMR, and two-dimensional images, pre-destruction images, post-destruction images of the kidney during the experiment. After using MB_{C4d}, the US signals detected in the renal allograft with AMR were significantly higher than that of the control group. Signals of the syngeneic renal graft administered with MB_{C4d} and the renal allograft administered with MB_{Con} were not significantly different. FIG. 5B: normalized intensity differences (NIDs) were calculated using the destruction-replenishment approach. (n=5, * * * p<0.001, d3: the 3rd day after operation).

FIG. 6A and FIG. 6B are an acute antibody-mediated cardiac allograft rejection model established by performing cardiac transplantation 2 weeks after skin transplantation. FIG. 6A: changes in the levels of donor-specific antibodies (IgG and IgM) after skin transplantation. FIG. 6B: histological evaluation of the cardiac allograft 3 days after transplantation. Hematoxylin and eosin staining revealed interstitial vasculitis and hemorrhage. C4d staining revealed extensive C4d deposition. The syngeneic cardiac graft served as a control. (* P<0.05, ** P<0.01, *** P<0.001. ST: skin transplantation; CT: cardiac transplantation).

FIG. 7A and FIG.7B include C4d-targeted ultrasound (US) images and normalized intensity differences (NIDs) of different groups. FIG. 7A: representative targeted ultrasound (US) images generated using C4d-targeted microbubbles (MB_{C4d}) and control microbubbles (MB_{Con}) in a cardiac allograft with antibody-mediated rejection (AMR). MB_{C4d} were used as a control in syngeneic cardiac graft imaging. In the cardiac allografts with AMR, the US signals detected in the cardiac allograft group employing MB_{C4d} were significantly higher than those generated with MB_{Con} and those generated with MB_{C4d} in syngeneic cardiac graft . FIG. 7B: normalized intensity differences (NIDs) were calculated using the destruction-replenishment approach (n=4, * * * p<0.001. d3: the 3rd day after operation).

### DETAILED DESCRIPTION OF ILLUSTRATED EMBODIMENTS

The invention will be further illustrated with reference to the embodiments but are not limited thereto.

I. Research Method of the Present Invention

1. Preparation of microbubbles and in vitro experiments

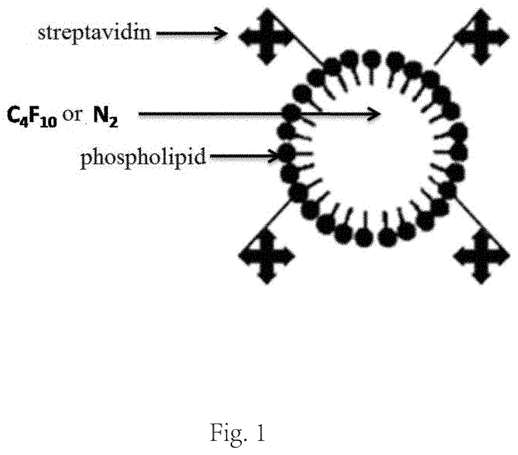

Commercially available streptavidin-coated microbubbles (MicroMarker™ Target Ready) were purchased from VisualSonics Inc. The product was a powder contained in a vial. It was ready to use after being dissolved in 1 mL of saline; the mean diameter of the microbubbles was 1.3 μm, the concentration of the microbubbles was 2×10⁹ per vial. After C4d antibodies (anti-Rat C4d Cat. No. HP8034; Hycult Biotech Inc., Plymouth Meeting, Pa.) were biotinylated, they can conjugate with streptavidin microbubbles. Biotinylated isotype-matched rabbit control immunoglobulin G (IgG) antibodies (Jackson ImmunoResearch Laboratories, West Grove, Pa.) was used as a specific control. Two types of microbubbles (C4d-targeted MBs [MB_{C4}d] and control MBs [MB_{Con}]) were prepared according to the manufacturer's instruction. MicroMarker™ Target Ready comprises a shell composed of phospholipids and streptavidin; perfluorocarbon (C₄F₁₀) or nitrogen (N₂) is encapsulated in the shell (FIG. 1). The microbubble shell can be synthesized from a phospholipid; it can also consist of a protein, a lipid, or a polymer. Apart from C₄F₁₀ and N₂, other inert gases, such as octafluoropropane (C₃F₈) and sulfur hexafluoride (SF₆), etc., can also be used as the gas core. These microbubbles can be linked to ligands through covalent linking or thiol-maleimide chemistry. The ligands then conjugate with vascular endothelial targets in situ to achieve a targeted action. When commercially available avidin-coated microbubbles were used, targeted microbubbles can be readily produced by adding an appropriate amount of biotinylated antibodies. Additionally, the current microbubbles designed are generally 1 μm to 10 μm in diameter and are similar in size to red blood cells. As a result, they can pass through the finest capillaries. Microbubbles that are too large are unstable in the circulation and will be quickly removed. On the other hand, microbubbles that are too small are likely to adversely affect imaging quality. Therefore, the diameter of most microbubbles was designed to be between 1 μm and 4 μm. The concentration of microbubbles refers to the number of microbubbles contained in 1 mL of a solution. Different microbubbles are produced in different sizes and numbers. Therefore, different volumes of microbubbles were used in detection. The solvent was mostly PBS or saline. Depending on different experimental animals used or different production processes of the microbubbles, different concentrations of microbubbles may be required. To much injection may cause ultrasonic attenuation and affect the observation and results, whereas if the concentration is too low, the targeted microbubbles may not conjugate effectively. The concentration range used in the literature is mostly between 10⁷ to 10⁹. We tested between 10⁶ to 10⁹ and asserted that the current concentration of 10⁸ to 10⁹ is the optimal concentration. Briefly, 1 mL of saline was injected into a vial containing 50 μg of antibodies, and the mixture was injected into a MicroMarker™ Target Ready vial (being exposed to the shear force of the flowing blood, targeted microbubbles conjugated with antibodies should usually have a ligand density of >50,000 antibodies per microbubble. The recommendation from a targeted microbubbles product manual is to mix 1 mL of targeted microbubbles with 20 μg of biotinylated antibodies. The method adopted by us was to add a supersaturated amount of 50 lug antibodies to fully conjugate to the targeted microbubbles; then, the unconjugated antibodies in excess were removed by elution), and the vial was incubated at room temperature for 20 minutes. The incubation process was accompanied by gentle shaking. The ligand that was not conjugated with microbubbles was removed by centrifugal washing. In subsequent animal experiments, 300 uL of dissolved microbubbles labeled with antibodies were used per animal in renal and cardiac allograft recipients.

FITC-labeled C4d antibodies were used as a glowing indicator for ligand binding to microbubble surfaces. A FACSCalibur flow cytometer was used to evaluate the fluorescence of FITC-C4d antibodies, the combination rate of streptavidin microbubbles and biotinylated antibodies was evaluated by fluorescence microscopy.

2. Establishing a Model of Antibody-Mediated Acute Renal and Cardiac Rejection.

Adult male (200 g to 250 g) Lewis and Brown Norway rats were purchased from Charles River Laboratories and housed in an animal room at Sun Yat-sen University. The animal experiments were reviewed and approved by the Institutional Animal and Use Committee of Sun Yat-sen University. Two weeks before renal and cardiac transplantation, the skin of a BN rat was transplanted onto the dorsal area of a Lewis rat. In renal transplantation, for every pre-sensitized Lewis recipient, the right kidney was removed and the left kidney remained before receiving donor BN renal transplantation. Briefly, the aorta, renal vein, and ureter of the renal graft were anastomosed to the aorta, inferior vena cava, and ureter of the recipient, respectively. In cardiac transplantation, the aorta and pulmonary artery of the donor heart were anastomosed to the aorta and inferior vena cava of the recipient, respectively. Ultrasound imaging was performed 3 days after renal and cardiac transplantation.

3. Detection of Circulating Donor Specific Antibodies

Serum samples were obtained from Lewis recipients at an indicated time. Circulating donor-specific IgG and IgM antibodies were estimated by flow cytometry. In short, recipient sera were incubated with BN donor splenocytes at 37° C. for 30 min, washed, and then incubated with FITC-labeled anti-rat IgG (Abcam, Cambridge, England) and rhodamine red-conjugated anti-rat IgM (Jackson ImmunoResearch Laboratories, West Grove, Pa.) at 4° C. for 1 h. The cells were analyzed by flow cytometry; the mean fluorescence intensity obtained was used to compare individual serum anti-donor antibody levels.

4. Histology and Immunohistology

To avoid the antigen blocking effect of the anti-C4d antibodies carried by the targeted microbubbles, we performed a histological examination on another group of rats. A rat AMR model was established according to the method described above. Renal and cardiac allografts were harvested on the 3rd day after operation. The original left kidney and the original heart retained during the transplantation served as a control group. Grafts were then formalin-fixed and embedded in paraffin before staining with hematoxylin and eosin (H&E), periodic acid-Schiff (PAS), anti-C4d (anti-Rat C4d Cat. No. HP8034; Hycult Biotech Inc., Plymouth Meeting, Pa.). Histologic changes and C4d staining in interstitial vascular tissue were examined by light microscopy.

5. Image Acquisition

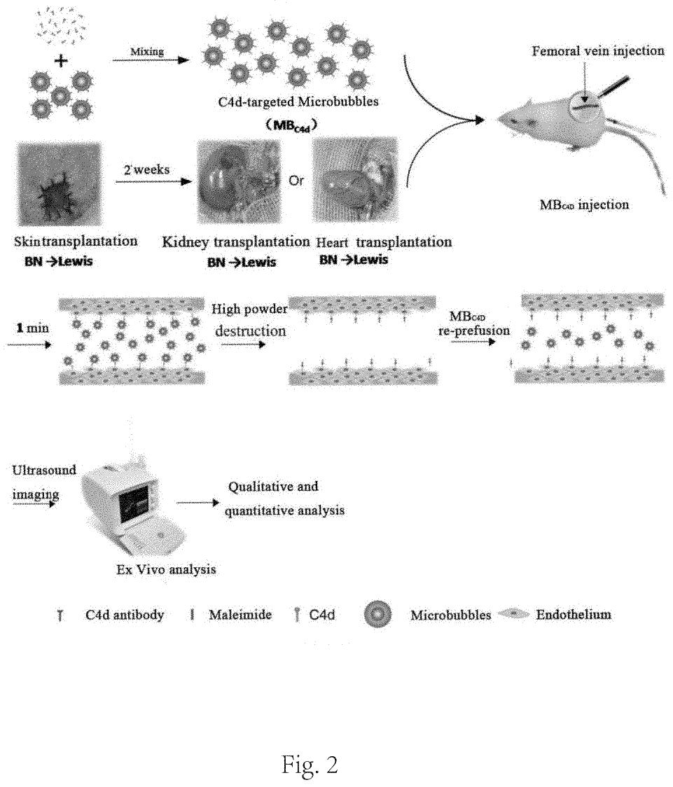

CEUS was performed using a clinical US imaging system (Logiq E9 digital premium ultrasound system, GE, Milwaukee, Wis.) and images of rat renal and cardiac allografts were collected using a broadband ML6-15D high-frequency scope with the following imaging parameters: frequency of 10 MHz, gain of 20-40 dB, image depth of 2-3 cm, acoustic output of 9%, dynamic range of 65 dB, and mechanical index of 0.09, at a long axis view of the renal allograft; frequency of 10 MHz, gain of 20-30 dB, image depth of 2-3 cm, acoustic output of 9%, dynamic range of 45 dB, and mechanical index of 0.09 at transection of the cardiac allograft. Targeted ultrasound imaging was performed via a destruction-replenishment approach (FIG. 2); specifically: renal and cardiac transplantation were performed 2 weeks after skin transplantation. Meanwhile, streptavidin-labeled microbubbles were conjugated with biotinylated anti-C4d antibodies to produce C4d-targeted microbubbles (MB_{C4d}). On the 3rd day after transplantation, MB_{C4d} was injected via the femoral vein of the recipients, and ultrasound imaging was performed. One minute after the intravenous administration, a signal generated by microbubbles attached to C4d was recorded as the first data, said microbubbles include microbubbles attached to the vascular lumen and free circulating microbubbles. Within the beam height of the ultrasonic transducer, the power of ultrasonic pulse was increased to uniformly destruct the microbubbles attached to the tissue and the free circulating microbubbles. After that, the free circulating microbubbles were replenished until reaching the image saturation level. At 10 seconds after destruction, a second ultrasound imaging was performed; the ultrasonic signal at this point was recorded as the second data. The ultrasonic signal for the microbubbles bound to C4d is derived from the difference of microbubble numbers before and after the destruction pulse in the image. CEUS qualitative analysis software IDS and quantitative analysis software Sonamath were used to quantitatively and qualitatively analyze the ultrasonic signal for the microbubbles bound to C4d (apart from the destruction-replenishment approach, there are many other approaches for quantitative targeted ultrasound imaging, such as observing the degree of contrast intensity at the same time, observing the imaging time, etc.). In short, all animals were injected with both MB_{Con} and MB_{C4d} via the femoral vein, an ultrasound probe was fixed in the area to be observed; continuous imaging was performed for 1 minute, during which a sufficient number of microbubbles (including bound and free microbubbles) were observed to enter the tissue, and pre-destruction images were obtained. The mechanical index was increased from 0.07 to 0.24 for 1 second by setting a “flash” function, thereby eliminating all the microbubbles in the observation area. Subsequently, continuous imaging was performed for 10 seconds, during which free MBs were observed to re-enter the circulation, and post-destruction images were obtained. To ensure the clearance of microbubbles from the previous study in the circulation and to prevent these microbubbles from interfering with the next study, the study of C4d targeted MBs was repeated 20 minutes after the study of control MBs.

6. Qualitative and Quantitative Analysis of Targeted Ultrasound Imaging

Qualitative and quantitative targeted US imaging signals from MBs that were bound to C4d were respectively analyzed using the CEUS qualitative analysis software IDS and quantitative analyzing software Sonamath (AmbitionT.C., Chongqing, China). In the region of interest, information in the image obtained by a contrast frame before destruction corresponded to both MBs bound to a target and MBs not bound to a target in the blood, while the contrast frame after destruction only indicated the freely circulating MBs. By subtracting the post-destruction signal from the pre-destruction signal, the imaging signal generated by MBs bound to the target in situ can be qualitatively analyzed. The quantification of targeted US imaging signal can be achieved by calculating normalized intensity differences (NIDs [%]=(pre-destruction signal intensity−post-destruction signal intensity/pre-destruction signal intensity). That is, the ratio of the attached MBs imaging signal intensity to the total MBs imaging signal intensity was calculated.

7. Statistical Methods

The data software SPSS 20.0 was used for statistical analysis. All values were expressed as the mean±standard deviation. A T-test or a one-way ANOVA was used for analysis. Significance was assumed when p<0.05.

II. Research Results

1. Characterization of Microbubbles

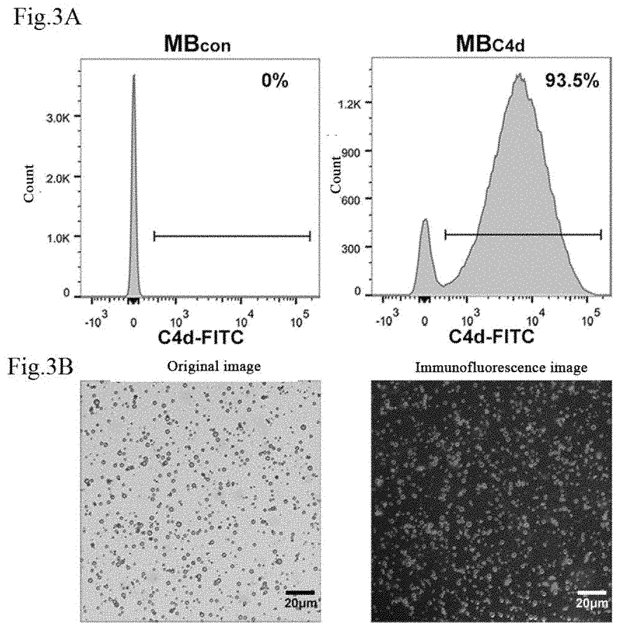

The mean diameter of the targeted microbubbles was 1.3 μm, and the concentration of microbubbles was 2×10⁹ per mL. After the biotinylated anti-C4d antibodies were mixed with FITC, flow cytometry showed that the binding rate of the microbubbles to the C4d antibodies was as high as (93% ±4.5%) (FIG. 3A). Furthermore, the successful coupling of the C4d to the surface of microbubbles was verified by fluorescence microscopy (FIG. 3B).

2. Establishing an Antibody-Mediated Renal Allograft Rejection Model

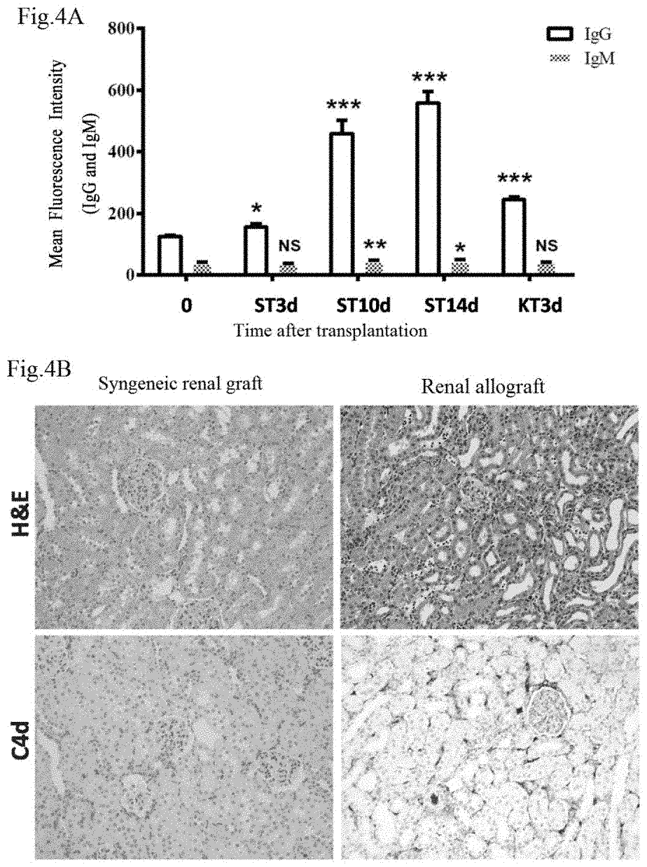

We used a pre-sensitization method by skin grafting to establish an antibody-mediated renal allograft rejection rat model. After skin transplantation, we monitored the levels of donor-specific antibodies IgG and IgM produced by the recipient, the levels were found to gradually increase over time. At two weeks after skin transplantation, IgG level was significantly higher than its normal level (558.2±81.7 vs 125.3±10.6), whereas IgM level was slightly higher than its normal level (47.0±6.1 vs 40.7±2.2) (FIG. 4A). At this point, renal transplantation was performed to establish the antibody-mediated renal allograft rejection model. On the 3rd day after renal transplantation, we studied the histological features of the renal allograft, and features including interstitial vasculitis, hemorrhage and edema, and tubular necrosis were observed. Additionally, extensive C4d deposition was also detected in the renal allograft (FIG. 4B). These features conformed to the Banff criteria of AMR. In a renal allograft acting as a control, it is possible to not observe any AMR pathological features and C4d deposition (FIG. 4B).

3. Imaging and Quantitative Analysis of Renal Grafts by C4d Targeted Contrast-Enhanced Ultrasound

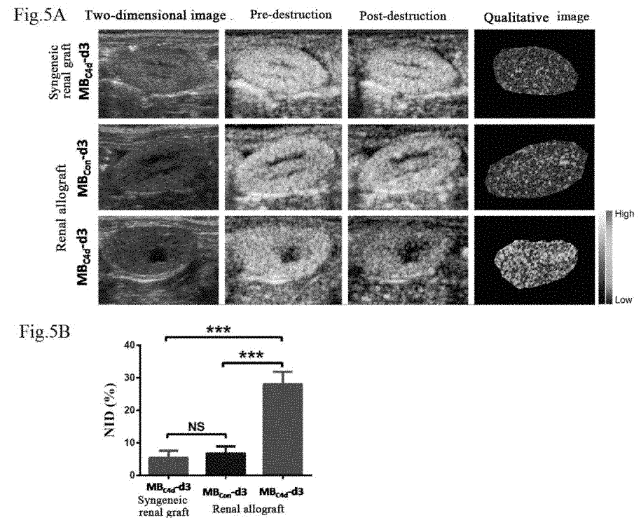

To obtain targeted ultrasound image information, MB_{Con} and MB_{C4d} were injected into rats via the femoral vein. The first image and the second image data were acquired according to the experimental protocol, and then qualitative images of the C4d targeted microbubbles were obtained by the CEUS qualitative analysis software IDS. As shown in FIG. 5A, in the images of the renal grafts, compared with the MB_{Con} group and the syngeneic renal graft control group, the MB_{C4d} renal allograft group produced stronger molecular ultrasound imaging signals. The signal intensities of the syngeneic renal graft group and the MB_{Con} group were similar. Furthermore, NID was used as a parameter for the quantitative analysis of C4d-targeted ultrasound imaging. It was found that the NID value of the renal allograft MB_{C4d} group was significantly higher than those of the MB_{Con} group and the syngeneic renal graft group (28.0% ±3.8% vs 6.7% ±2.2% and 5.4% ±2.2%), but there was no significant difference between the syngeneic renal graft group and the MB_{Con} group (FIG. SB).

4. Antibody-Mmediated Cardiac Allograft Rejection Model

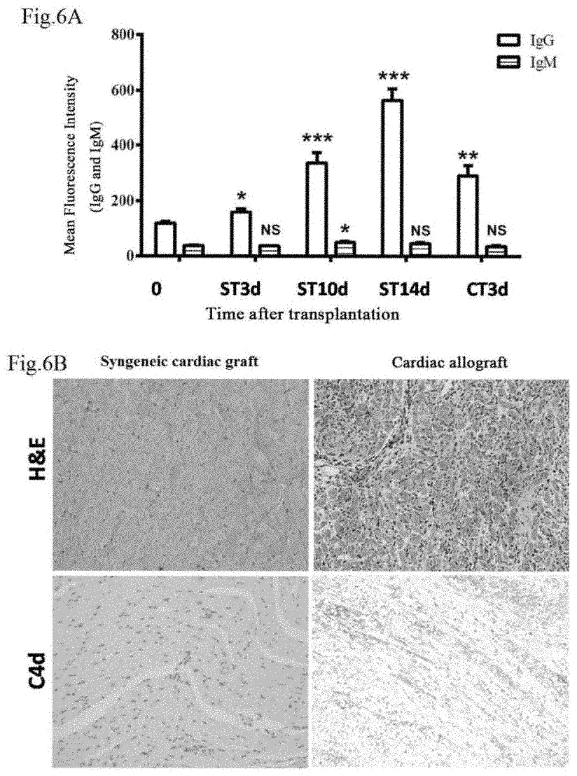

Similar to the method used to establish the antibody-mediated renal allograft rejection rat model, two weeks after pre-sensitization with skin transplantation. The DSA level was monitored and once it reached a peak, cardiac transplantation was performed (FIG. 6A). On the 3rd day after cardiac transplantation, a tissue specimen of the graft was collected for pathological examination, and histological features including microcirculation inflammation, edema, and endothelial cell proliferation were observed. Diffuse C4d deposition was also detected in the capillaries of the cardiac allograft (FIG. 6B). These features conformed to the ISHLT criteria of AMR. A syngeneic cardiac graft was used as the control, and there was no AMR-related pathology.

5. Imaging and Quantitative Analysis of Cardiac Grafts by C4d Targeted Contrast-Enhanced Ultrasound

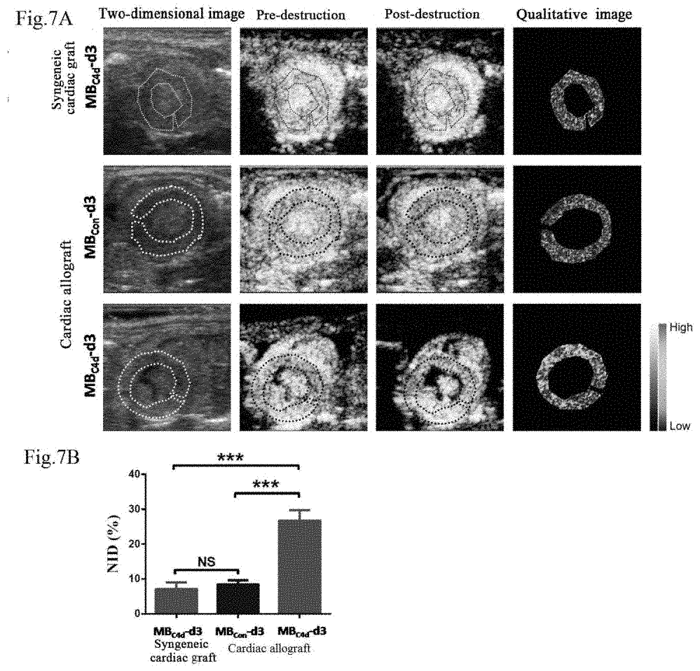

Identical to the method for the analysis of renal grafts, on the 3rd day after cardiac transplantation, MB_{Con} was first injected through the femoral vein, and MB_{C4d} was then injected after an interval. The first image and the second image data were acquired according to the experimental protocol, and then qualitative images of the C4d-targeted microbubbles were obtained by the CEUS qualitative analysis software IDS. As shown in FIG. 7A, in the cardiac grafts, it can be observed that the MB_{C4d} group displayed stronger molecular ultrasound imaging signals than the MB_{Con} group and the syngeneic cardiac graft group. Furthermore, quantitative analysis showed that the NID value of the MB_{C4d} group was significantly higher than those of the MB_{Con} group and the syngeneic cardiac graft group (26.7% ±3.0% vs 8.4% ±1.2% and 7.1% ±2.0%) (FIG. 7B), but there was no significant difference between the MB_{Con} group and the syngeneic cardiac graft group.

In this study, we confirmed for the first time that targeted ultrasound can be applied in the qualitative and quantitative analysis of C4d in renal and cardiac allografts, allowing the non-invasive diagnose of AMR.

It has been recognized that AMR was the primary cause of renal allograft dysfunction. Furthermore, in cardiac transplantation, the occurrence of AMR is also considered to be closely related to the progression of graft vascular disease and poor prognosis. Regardless of cardiac or renal transplantation, the current diagnosis of AMR requires a tissue biopsy, the risks associated with invasive examination cannot be avoided. Therefore, there is an urgent need for a non-invasive and quantifiable detection method. A prominent advantage of targeted ultrasound imaging lies in its non-invasiveness and its quantifiability. C4d plays an important role in AMR diagnosis. Its characteristic distribution in interstitial blood vessels suggests that C4d may be a key breakthrough point to achieve non-invasive AMR diagnosis.

With the invention of targeted ultrasound contrast agents, ultrasound imaging has been included in the field of molecular imaging research. Compared with other imaging methods, such as computed tomography (CT), nuclear medicine, X-ray, and angiography, targeted ultrasound has demonstrated some advantages clinically because it is both economical and convenient. In particular, it allows real-time and effective observation of structure and function at a gross anatomical level and molecular level. Many laboratories had attempted to use targeted CEUS to detect acute renal or cardiac rejection. Grabner et al. used microbubbles targeting CD3, CD4, and CD8 to diagnose acute rejection in renal allografts, but a limitation of this study is that it cannot distinguish between acute rejection mediated by cells and by antibodies. In addition, there are two other studies on the diagnosis of acute cardiac rejection using targeted ultrasound, in which microbubbles targeting leukocytes and ICAM-1 were respectfully employed.

However, leukocyte infiltration and high ICAM-1 expression not only occur in acute rejection, but also in tissue ischemia-reperfusion injury (IRI). In addition, identical symptoms may also appear in urinary tract infections and BK virus-associated nephropathy, yet these studies do not distinguish between these diseases. More notably, none of the previous studies can be used to diagnose AMR.

Only when AMR occurs, C4d is deposited in the interstitial blood vessels of renal and cardiac allografts. As a result, it is easy to distinguish between cell-mediated rejection, IRI, and inflammatory diseases. C4d becomes the most ideal target for targeted CEUS in the diagnosis of AMR because of this high specificity.

Similar to other studies, the skin of donor BN rats was transplanted to a Lewis recipient for pre-sensitization two weeks before renal transplantation and cardiac transplantation in rats. After skin transplantation, the donor-specific antibodies (DSA) produced by the recipient was detected, and the level of IgG antibodies was found to increase significantly. Two weeks later, DSA level increased significantly. At this point, kidney and cardiac transplantations were performed separately, and histopathological specimens of the allografts were collected three days later. Interstitial vasculitis, hemorrhage, and diffuse C4d deposition were observed in the allografts, which conformed to the Banff criteria of AMR.

Attaching ligands to microbubbles through biotin-avidin binding is the most common method to produce targeted microbubbles. The microbubbles coated with streptavidin used in this study were purchased from VisualSonics Inc. The specific method of linking C4d with biotin was described above. After mixing biotinylated C4d antibodies with microbubbles coated with avidin, the binding rate was above 90%, confirming successful preparation of C4d-targeted microbubbles.

The destruction-replenishment approach was used to calculate the signal intensity of the targeted ultrasound image. In a qualitative analysis, a software, IDS, was employed to generate a qualitative image through analyzing the difference between a first signal obtained before destruction and a second signal obtained after destruction. Such a qualitative image is more intuitive. NID (NID=(pre-destruction signal intensity−post-destruction signal intensity)/pre-destruction signal intensity) was used for quantitative analysis. It significantly reduces errors caused by individual differences.

Qualitative analysis of C4d in renal and cardiac allografts with acute AMR was carried out using targeted ultrasound imaging. The signals produced by the MB_{C4d} group were significantly stronger than those produced by the MB_{Con} group and the syngeneic renal and cardiac graftgroup. Further quantitative analysis of C4d also showed that the NID value of the MB_{C4d} group was significantly higher than those of the two control groups. Through qualitative and quantitative analysis results, we confirmed that diagnosing acute renal and cardiac AMR through C4d-targeted ultrasound is indeed feasible and effective.

To conclude, C4d deposited in the interstitial vessels of renal or cardiac allografts can be qualitatively and quantitatively detected by C4d-targeted ultrasound imaging; this method can be used to diagnose acute antibody-mediated rejection. Clinical This method is expected to be applied to clinical practice to achieve the non-invasive diagnosis of AMR. C3d and C4d have the same significance in renal and cardiac allograft AMR and can both be used in the design of targeted microbubbles. Additionally, apart from renal and cardiac allografts with AMR, C3d and C4d are also expressed in hepatic allografts with AMR, certain autoimmune diseases, tumors, and kidney diseases. C4d-targeted microbubbles can also be used in a novel, non-invasive diagnosis of these diseases.

The aforementioned embodiments are only used to illustrate the technical solutions of the present invention. They are not intended to limit the scope of the present invention. Although the present invention has been described in detail with reference to the preferred embodiments, those skilled in the art will appreciate that variations, modifications or equivalent replacements are allowed within the scope of the invention, and such variations fall within the protection scope of the present invention.

1. A targeted microbubble, characterized in that the targeted microbubble comprises a microbubble composed of a shell and a gas encapsulated in the shell, the shell is conjugated with a C4d antibody or a C3d antibody.

2. The targeted microbubble according to claim 1, characterized in that the shell is coated with streptavidin, the C4d antibody is a biotin-labeled antibody.

3. The targeted microbubble according to claim 1, characterized in that a surface of the shell is coated with streptavidin, the C3d antibody is a biotin-labeled antibody.

4. The targeted microbubbles according to claim 1, characterized in that the C4d antibody or the C3d antibody is a fluorescence-labeled antibody.

5. The targeted microbubble according to claim 1, characterized in that a diameter of the targeted microbubble is 1 μm to 10 μm.

6. The targeted microbubble according to claim 1, characterized in that the shell comprises at least one selected from the group consisting of a phospholipid, a protein, a lipid, and a polymers; the gas comprises at least one selected from the group consisting of perfluorocarbon, nitrogen, octafluoropropane, and sulfur hexafluoride.

7. A method for preparing the targeted microbubble according to claim 2, characterized in that the method comprises mixing and incubating a microbubble with a biotin-labeled C4d antibody or a biotin-labeled C3d antibody to obtain the targeted microbubble; the microbubble comprises a shell coated with streptavidin.

8. Use of the targeted microbubble according to claim 1 as a contrast agent in preparation of a diagnostic reagent or a diagnostic reagent kit for AMR in a renal allograft, AMR in a cardiac allograft, AMR in a hepatic allograft, an autoimmune disease, cancer, or a kidney disease.

9. A system for diagnosing AMR in a renal allograft, characterized in that the system comprises:
a data-inputting module for inputting first ultrasound intensity data and second ultrasound intensity data into a model calculation module; wherein an ultrasonic signal generated by a microbubble attached to a vascular lumen through C4d or C3d binding and a free circulating microbubble is recorded as the first ultrasonic intensity data; within a beam height of an ultrasonic transducer, power of an ultrasonic pulse is increased to uniformly destruct a microbubble attached to a tissue and the free circulating microbubble, the free circulating microbubble is replenished until reaching an image saturation level; at 10 seconds after destruction, a second ultrasound imaging is performed, an ultrasonic signal at this point is recorded as the second ultrasound intensity data;
the model calculation module comprises a normalized intensity difference model for calculating normalized intensity difference according to the first ultrasonic intensity data, the second ultrasonic intensity data, the normalized intensity difference NID=((the first ultrasonic intensity data−the second ultrasound intensity data)/the first ultrasound intensity data);
a result-outputting module, in which a NID value of a normal kidney is set as a control, the AMR is diagnosed when a NID value of a patient's renal allograft is significantly greater than the NID value of the normal kidney.

10. A system for diagnosing AMR in a cardiac allograft, characterized in that the system comprises:
a data-inputting module for inputting first ultrasound intensity data and second ultrasound intensity data into a model calculation module; wherein an ultrasonic signal generated by a microbubble attached to a vascular cavity through C4d or C3d binding and a free circulating microbubble is recorded as the first ultrasonic intensity data; within a beam height of an ultrasonic transducer, power of an ultrasonic pulse is increased to uniformly destruct a microbubble attached to a tissue and the free circulating microbubble, the free circulating microbubble is replenished until reaching an imaging saturation level, at 10 seconds after destruction, a second ultrasound imaging is performed, and an ultrasonic signal at this point is recorded as the second ultrasound intensity data;
the model calculation module comprises a normalized intensity difference model for calculating normalized intensity difference result according to the first ultrasonic intensity data, the second ultrasonic intensity data, the normalized intensity difference NID=((the first ultrasonic intensity data−the second ultrasound intensity data)/the first ultrasound intensity data);
a result-outputting module, in which a NID value of a normal heart is set as a control, the AMR is diagnosed when a NID value of a patient's cardiac allograft is significantly greater than the NID value of the normal heart.

11. A method for diagnosing AMR in a renal allograft, characterized in that the method comprises using the targeted microbubble according to claim 1 as a contrast agent for diagnosis.

12. The method according to claim 11, characterized in that the method comprises steps of:
(1) recording an ultrasonic signal generated by a microbubble attached to a vascular lumen through C4d or C3d binding and a free circulating microbubble as a first ultrasonic intensity data; increasing power of an ultrasonic pulse within a beam height of an ultrasonic transducer to uniformly destruct a microbubble attached to a tissue and the free circulating microbubble; then, replenishing the free circulating microbubble until reaching an image saturation level; performing a second ultrasound imaging at 10 seconds after destruction, recording an ultrasonic signal at this point as a second ultrasound intensity data;
(2) using a NID value of a normal kidney as a control, the AMR is diagnosed when a NID value of a patient' s renal allograft is significantly greater than the NID value of the normal kidney; wherein normalized intensity difference NID=((the first ultrasound intensity data−the second ultrasound Intensity data)/the first ultrasound intensity data).

13. A method for diagnosing AMR in a cardiac allograft, characterized in that the method comprises using the targeted microbubble according to claim 1 as a contrast agent for diagnosis.

14. The method according to claim 13, characterized in that the method comprises steps of:
(1) recording an ultrasonic signal that is generated by a microbubble attached to a vascular lumen through C4d or C3d binding and a free circulating microbubble as a first ultrasonic intensity data; increasing power of an ultrasonic pulse within a beam height of an ultrasonic transducer to uniformly destruct a microbubble attached to a tissue and the free circulating microbubble; then, replenishing the free circulating microbubble until reaching an image saturation level, performing a second ultrasound imaging at 10 seconds after destruction recording an ultrasonic signal at this point as a second ultrasound intensity data;
(2) using a NID value of a normal heart as a control, the AMR is diagnosed when a NID value of the patient's cardiac allograft is significantly greater than the NID value of the normal heart; wherein normalized intensity difference NID=((the first ultrasound intensity data−the second ultrasound Intensity data)/the first ultrasound intensity data).

15. Use of the targeted microbubble according to claim 2 as a contrast agent in preparation of a diagnostic reagent or a diagnostic reagent kit for AMR in a renal allograft, AMR in a cardiac allograft, AMR in a hepatic allograft, an autoimmune disease, cancer, or a kidney disease.

16. Use of the targeted microbubble according to claim 3 as a contrast agent in preparation of a diagnostic reagent or a diagnostic reagent kit for AMR in a renal allograft, AMR in a cardiac allograft, AMR in a hepatic allograft, an autoimmune disease, cancer, or a kidney disease.

17. A method for diagnosing AMR in a renal allograft, characterized in that the method comprises using the targeted microbubble according to claim 2 as a contrast agent for diagnosis.

18. A method for diagnosing AMR in a renal allograft, characterized in that the method comprises using the targeted microbubble according to claim 3 as a contrast agent for diagnosis.

19. A method for diagnosing AMR in a cardiac allograft, characterized in that the method comprises using the targeted microbubble according to claim 2 as a contrast agent for diagnosis.

20. A method for diagnosing AMR in a cardiac allograft, characterized in that the method comprises using the targeted microbubble according to claim 3 as a contrast agent for diagnosis.
